(19) United States
(12) Patent Application Publication
(10) Pub. No.: US 2020/0297798 A1
(43) Pub. Date: Sep. 24, 2020

(54) KEY AMINO ACID SITES REGULATING NUCLEAR EXPORT OF NUCLEOPROTEIN IN INFLUENZA A AND B VIRUSES AND USE THEREOF AS ANTI-INFLUENZA VIRUS DRUG TARGETS

(71) Applicant: INSTITUTE OF MICROBIOLOGY, CHINESE ACADEMY OF SCIENCES, Beijing (CN)

(72) Inventors: Wenjun LIU, Beijing (CN); Lei SUN, Beijing (CN); Weinan ZHENG, Beijing (CN); Jing LI, Beijing (CN); Limin YANG, Beijing (CN); Wenhui FAN, Beijing (CN)

(73) Assignee: INSTITUTE OF MICROBIOLOGY, CHINESE ACADEMY OF SCIENCES, Beijing (CN)

(21) Appl. No.: 16/645,227

(22) PCT Filed: Sep. 22, 2017
(86) PCT No.: PCT/CN2017/102856

§ 371 Date: Mar. 6, 2020

(30) Foreign Application Priority Data
Sep. 8, 2017 (CN) 201710804078.2

(51) Int. Cl. A61K 38/16; C12Q 1/70; G16B 5/00; A61K 31/405; A61P 31/16

(52) U.S. Cl. CPC A61K 38/162; C12Q 1/70; A61P 31/16; A61K 31/405; G16B 5/00

(57) ABSTRACT

Provided are key amino acid sites regulating the nuclear export of a nucleoprotein in influenza A and B viruses and the use thereof as anti-influenza virus drug targets. Also provided is a pharmaceutical composition for treating the influenza A or B virus infection, comprising a compound that regulates the nuclear export of a nucleoprotein in the influenza A or B virus.

### REFERENCES TO RELATED APPLICATIONS

This application claims the benefit of Chinese Patent Application No. 201710804078.2, filed to the State Intellectual Property Office of the People's Republic of China on Sep. 8, 2017, the entire content of which is hereby incorporated by reference in its entirety.

### FIELD OF THE INVENTION

The present application belongs to biomedical field, relates to key amino acid sites regulating the nuclear export of nucleoprotein in influenza A and B viruses, and use thereof as anti-influenza virus drug targets. The present application also relates to a pharmaceutical composition for treating influenza A or B virus infection, comprising a compound that regulates the nuclear export of nucleoprotein in the influenza A or B virus.

### BACKGROUND OF THE INVENTION

Influenza A and B viruses belong to the Orthomyxoviridae family. Influenza A viruses can infect humans and many animal species. Many subtypes of influenza A viruses have high morbidity and/or mortality, which seriously threaten the health of animals and human. Influenza B virus mainly infects humans. Although influenza B virus has relatively less morbidity in human, pneumonia caused by its infection often has high mortality. On the other hand, the surface proteins of influenza viruses are prone to mutation in antigenic sites, which make them being resistant to the drugs targeting the surface proteins of the influenza viruses. Therefore, how to design a drug using a core protein of influenza viruses as a target has received increasing attention.

The genetic material of both influenza A and B viruses is eight single-stranded negative sense RNAs. Viral RNA (vRNA) coated by nucleoprotein (NP) and viral polymerase (PA, PB1, and PB2) form viral ribonucleoprotein (vRNP) complex. vRNP is a basic functional unit for replication and transcription of viral genome RNA. After influenza virus is internalized into cells via the endosomal pathway, vRNP is released into the cytoplasm and transported into the nucleus. The replication of the viral genome and the transcription and translation of the viral protein occur in the nucleus, and newly synthesized NPs and the polymerases are assembled with vRNAs into progeny vRNPs. In the late phase of infection, the progeny vRNPs are exported from the nucleus to the plasma membrane, viral proteins are recruited and new virions are released via budding.

NP plays crucial roles in the nuclear import and export of vRNPs. NP in cytoplasmic vRNPs interacts with the nuclear transporter importin-α (IMPα) in host cell, and the cytoplasmic vRNPs are transported into the nucleus via the IMPα-IMPβ1 pathway. In the late phase of infection, NP in the progeny vRNPs in the nucleus binds to the influenza virus M1 protein, and the influenza virus NEP protein is used as an adaptor between vRNP-M1 complex and host nuclear export protein CRM1 (chromosome region maintenance 1) nuclear export pathway to export vRNP-M1 from the nucleus (Nature Reviews Microbiology 13, 28-41 (2015)).

For using NP as a drug target, researches have shown that in assembly of vRNPs of the influenza A virus, the binding of influenza A virus nucleoprotein (ANP) to vRNAs relies on the multiple amino acid sites in the RNA binding groove region of ANP (Nature 444: 1078-1082, 2006). As a result of identifying interaction between the binding groove region and existing small molecule libraries by MD modeling, naproxen and its derivatives can inhibit the replication of influenza A virus by inhibiting the binding of ANP to vRNAs. The results of further computational simulation have shown that the action targets of naproxen are the amino acids Y148, R152, R355, and R361 of ANP which are located in the RNA binding groove region (Lejal N, Tarus B, Bouguyon E, Chenavas S, Bertho N, Delmas B, Ruigrok R W, Di Primo C, Slama-Schwok A, Structure-based discovery of the novel antiviral properties of naproxen against the nucleoprotein of influenza A virus, Antimicrob Agents Chemother. 2013, 57 (5): 2231-42; Tarus B, Bertrand H, Zedda G, Di Primo C, Quideau S, Slama-Schwok A., Structure-based design of novel naproxen derivatives targeting monomeric nucleoprotein of Influenza A virus, J Biomol Struct Dyn. 2015; 33 (9): 1899-912.; and US2014/0163107A1). Naproxen is a class of nonsteroidal anti-inflammatory drugs, mainly for in clinical treating arthritis (such as rheumatic and rheumatoid arthritis, and osteoarthritis), ankylosing spondylitis, gout, tenosynovitis, etc., and relieving pain caused by musculoskeletal sprains, contusions and injuries, dysmenorrheal, or the like, and it has not been reported that this drug can be used for antiviral treatment.

In addition, current studies have not identified the key sites of influenza A virus NP involved in vRNP nuclear import/export, and there have been no report on the sites of influenza B virus NP (BNP) useful as a drug target.

### SUMMARY OF THE INVENTION

The present application directs to the key amino acid sites of nucleoprotein of influenza A and B viruses for nuclear import/export of vRNPs, and proposes for the first time that tyrosine at position 148 of influenza A virus nucleoprotein (ANP) and phenylalanine at position 209 of influenza B virus nucleoprotein (BNP) are the key sites for the nuclear export of vRNPs of influenza A and B viruses, respectively. The aforementioned sites can be used as drug targets to design and screen a drug which inhibits or blocks the nuclear export of the influenza virus vRNPs, for effectively treating influenza.

In the first aspect, the present application provides a method for treating a subject infected with influenza A virus, characterized in administering a therapeutically effective amount of a pharmaceutical composition to the subject, the pharmaceutical composition comprising a compound which inhibits interaction between tyrosine at position 148 (Y148) of influenza virus A nucleoprotein (ANP) and CRM1.

In the second aspect, the present application provides a method for inhibiting the nuclear export of influenza A virus nucleoprotein, characterized in inhibiting the interaction between Y148 of influenza A virus nucleoprotein and CRM1.

In the third aspect, the present application provides a method for inhibiting the nuclear export of influenza A virus nucleoprotein in a subject infected with influenza A virus, characterized in administering to the subject a compound that interacts with Y148 of influenza A nucleoprotein.

In the fourth aspect, the present application provides a medicament for treating a subject infected with influenza B virus, the medicament comprising a compound which inhibit the activity of influenza B virus nucleoprotein by interacting with phenylalanine at position 209 (F209) of influenza B virus nucleoprotein (BNP).

In the fifth aspect, the present application provides a method for treating a subject infected with influenza B virus, characterized in administering a therapeutically effective amount of a pharmaceutical composition to the subject, the pharmaceutical composition comprising a compound that interacts with F209 of BNP.

In the sixth aspect, the present application provides a method for inhibiting the nuclear export of influenza B virus nucleoprotein, characterized in blocking the interaction between F209 of influenza B virus nucleoprotein and CRM1.

In the seventh aspect, the present application provides a method for inhibiting influenza B virus infection in a subject, characterized in administering to the subject a compound that interacts with F209 of influenza B virus nucleoprotein.

In the eighth aspect, the present application provides use of F209 of influenza B virus nucleoprotein as a target for an anti-influenza B virus medicament.

In the ninth aspect, the present application provides a method for screening a compound having an effect of inhibiting influenza B virus infection, which comprises the following steps:

(a) modeling the 3-dimensional structure of influenza B virus nucleoprotein using a 3-dimensional molecular structure modeling software;
(b) screening a candidate compound capable of interacting with phenylalanine at position 209 of influenza B virus nucleoprotein using a molecular docking software; and
(c) verifying whether the candidate compound can inhibit replication of influenza B virus at a cellular level;
wherein, the sequence of influenza B virus nucleoprotein is PDB ID: 3TJ0 in the PDB database.

In the tenth aspect, the present application provides a compound screened by the method of the ninth aspect.

### BRIEF DESCRIPTION OF THE DRAWINGS

FIG. 1 shows the results of the cellular location of the wild-type ANP (A-WT), A-Y148A mutant protein, and A-Y148F mutant protein at 12, 24, and 36 hours post transfection. NP shows the TRITC channel fluorescence, indicating the location of ANP, A-Y148A or A-Y148F protein. The merged images (Merge) are the results of merging TRITC and DAPI channels (indicating nuclear location). p.t. represents post transfection.

FIG. 2 shows the results of western blot of the interaction between the wild-type ANP (A-WT), A-Y148A mutant protein, or A-Y148F mutant protein and importin-α1 (A) and CRM1 (B). Input: loading control. IP: FLAG: results of western blot with pulldown treatment of anti-FLAG M2 beads (i.e., co-immunoprecipitation).

FIG. 3 shows the growth curves of the rescued influenza A viruses in which nucleoprotein is respectively the wild-type ANP (A-WT) and the mutant A-Y148F, according to plaque assay. Error bars represent the standard deviation of three independent experiments.

FIG. 4 shows the results of the effect of A-Y148F mutation on the replication and pathogenicity of virus in mice. (A) Changes in body weight of mice over days post infection. (B) Changes in survival rate of mice over days post infection. (C) Changes in lung index of mice over days post infection. (D) Changes in virus titers over days post infection. (E) Lung appearance of mice 5 days post infection. A-WT is a positive control infected with the wild-type virus. A-Y148F is an experimental group infected with a virus having ANP with Y148F mutation. PBS is blank control. Error bars represent standard deviation of three independent experiments.

FIG. 5 shows a comparison of influenza A NP (ANP) and influenza B NP (BNP) in structure. (A) Schematic diagram of the structure of ANP (PDB ID NO: 2IQH) and BNP (PDB ID NO: 3TJ0) based on the structure of PDB, with the aromatic amino acid residues Y148 and F209 in the RNA binding grooves enriching basic amino acids specially marked. (B) Sequences adjacent to the residue Y148 of ANP and the residue F209 of BNP. Asterisks show amino acid residues having homology and identity.

FIG. 6 shows cell location results of the wild-type BNP (B-WT) (A) and B-F209A mutant protein (B) 12, 24, and 36 hours post transfection. NP shows TRITC channel fluorescence, indicating the location of BNP and B-F209A proteins. The merged images (Merge) are the results of the merge of TRITC and DAPI channels (indicating nuclear location).

FIG. 7 shows western blot results of interaction between the wild-type BNP (B-WT) or the mutant protein B-F209A and importin-α1 (A) and CRM1 (B). Input: loading control. IP:FLAG: western blot results of the group treated with anti-FLAG M2 bead pulldown (i.e. co-immunoprecipitation).

FIG. 8 shows effects of naproxen on the growth curves of the rescued influenza A virus (A) and the rescued influenza B virus (B) according to plaque assay. Error bars represent standard deviation of three independent experiments.

FIG. 9 shows fluorescence microscopy results of the effects of naproxen on the cell localization of the wild-type ANP (A) and BNP (B). +NPX is the experimental results with the addition of naproxen in the culture dishes. −NPX is control result without naproxen. NP shows TRITC channel fluorescence, indicating the localization of nucleoprotein. DAPI channel fluorescence indicates nuclear localization. The merge results of TRITC and DAPI channels are displayed in the merged images (Merge).

FIG. 10 shows western blot results of naproxen affecting interaction between the wild-type ANP (A) or the wild-type BNP (B) and CRM1. Input: loading control. IP: aFLAG: western blot results of the group treated with anti-FLAG M2 bead pulldown (i.e. co-immunoprecipitation).

FIG. 11 shows the results of the effects of naproxen on replication and pathogenicity of influenza A and B viruses in mice. (A-B) Changes in the body weight of mice after influenza A virus infection (A) or influenza B virus infection (B) over days post infection. (C-D) Survival rates of mice after influenza A virus infection (C) or influenza B virus infection (D) over days post infection. (E) Lung appearance of mice 5 days post infection. (F-G) Changes in lung index of mice after influenza A virus infection (F) or influenza B virus infection (G) over days post infection. (H-I) Changes in virus titer after influenza A virus infection (H) or influenza B virus infection (I) over days post infection. PBS is the blank control group. fluA/B is the control group in which PBS is injected intraperitoneally one day after influenza A or B virus infection. fluA/B+Ose is the treatment group with gastric perfusion of 1 mg oseltamivir one day after virus infection. fluA/B+NPX50 is the treatment group with intraperitoneal injection of 50 mM naproxen one day after virus infection. fluA/B+NPX5 is the treatment group with intraperitoneal injection of 5 mM naproxen one day after virus infection. Error bars represent standard deviation of three independent experiments.

### DETAILED DESCRIPTION

As mentioned above, the present invention directs to the key amino acid sites of influenza A and B virus nucleoprotein for the nuclear import/export of vRNPs, and it is found that a mutation at the Y148 position of influenza A nucleoprotein or the F209 position of influenza B virus nucleoprotein allows the nuclear export of influenza virus to be inhibited. Furthermore, the inventors found that naproxen and its derivatives act on the above sites to inhibit the nuclear export of NP. This influenza virus nuclear export mechanism can be used to design or screen a new antiviral medicament.

As used herein, the terms “influenza A virus” and “influenza B virus” have well-known meanings in the art, and such classification is based on antigenicity of influenza virus nucleoproteins. In terms of mutation ability, influenza A virus has a stronger variability than influenza B virus, and in particular, it has very high the amino acid mutation rate in hemagglutinin antigen and/or neuraminidase antigen on the surface, resulting in occurrence of cross-species transmission and failure of antiviral medicaments.

Nucleoprotein (NP) of influenza virus is highly expressed during viral infection and has multiple functions. As mentioned above, NP covers 8-segment genomic RNA and is assembled with 3 polymerases into viral ribonucleoprotein (vRNP) complex which controls viral transcription and replication. Studies on influenza A virus NPs have shown that NPs are highly conserved among different types of influenza A viruses (the amino acid sequences have approximately 90% identity), and there is no corresponding protein in cells. Therefore, nucleoprotein is a potential candidate target for antivirus (Antimicrobial Agents and Chemotherapy, 2013, Vol. 57 No. 5, pp. 2231-2242).

Influenza B virus nucleoprotein (BNP) and influenza A virus nucleoprotein (ANP) share many similarities in structure. However, in terms of sequence, there at least exists the following differences between BNP and ANP: (i) BNP contains a significant extended N-terminal region (amino acid positions 1-70); (ii) the nuclear localization sequences NLS-1 and NLS-2 in ANP are not present in BNP; (iii) some known RNA binding regions in ANP, in particular the flexible basic loop at positions 74-88 is not conserved in BNP; (iv) the linkers and tailing loops involved in NP homooligomerization are not conserved; and (v) the alignment of the C-terminus of ANP and BNP sequences appears gaps. In addition, it has been revealed by sequence alignment that BNP also has positively-charged RNA binding groove corresponding to the RNA binding groove of ANP, in which two positively-charged residue clusters are G1 (K125, K126, R235, R236) and G2 (R211, K213, R217) respectively. In addition, the region at positions 209-211 is a loop connecting each of the domains (J Virol. 2012, 86 (12): 6758-6767). In an embodiment of the present application, ANP can have, for example, the amino acid sequence of NCBI ID NO: ACF54602.1 or an amino acid sequence having 90% or more identity to the sequence. BNP can have, for example, the amino acid sequence of NCBI ID NO: AAA82969.1 or an amino acid sequence having 90% or more identity to the sequence.

As mentioned above, current studies have reported the important role of NP in the nuclear export of vRNPs. The first step of the transport of the progeny vRNPs from the nucleus to the plasma membrane is the nuclear export of vRNPs. Influenza A virus implements nucleus-to-cytoplasm transport by the CRM1 (also known as exportin-1)-dependent nuclear export pathway. Specifically, after vRNPs are released from chromatin, vRNPs are released from the nucleus by “daisy chain” complex, wherein viral nuclear export protein (NEP) serves as a adaptor between vRNP-M1 and CRM1RAN-GTP. On the cytoplasmic side of the nuclear pore complex (NPC), transport protein and vRNPs are released into the cytoplasm by RAN-GTP hydrolysis of RAN GTPase-activating protein (RAN GAP). In addition, in vitro experiments have shown that NP protein can still achieve nuclear export in the absence of other viral proteins; and NP can directly bind to CRM1 in vitro. These studies indicate that NP can mediate vRNP nuclear export through direct interaction with CRM1. However, the contribution of nuclear export signal motif of NP to vRNP nuclear export is unclear. In this regard, the role of NP in vRNP nuclear export is still unclear (Nature Reviews Microbiology 13, 28-41 (2015)). In an embodiment of the present application, CRM1 may be CRM1 of any host species capable of being infected with influenza A or B virus, depending on the subject. For example, the CRM1 protein can have the amino acid sequence of NCBI accession No. CAA69905.2, or an amino acid sequence having 90% or more identity to the sequence.

In the present application, the compound capable of interacting with tyrosine at position 148 of influenza A virus nucleoprotein or phenylalanine at position 209 of influenza B virus nucleoprotein to inhibit or block the interaction between nucleoprotein and CRM1 is for example naproxen or a derivative thereof. It is known in the art that naproxen is the common name of (+) α-methyl-6-methoxy-2-naphthaleneacetic acid. The derivatives of naproxen share the common structure of 2-arylpropionic acids. The known derivatives of naproxen in the art include Naproxen Sodium, DL-Naproxen, naproxen methyl ester, 23979-41-1, Naproxcinod, Naproxen-ETEMESIL, naproxen ethyl ester, Naproxen Glucuronide, 5-chloro naproxen, and 5-iodo naproxen, etc. The term “subject” as used herein refers to a patient in need of treatment of influenza A or B virus infection in the body. The subject can be human or animal, in particular mammal or avian. For example, the subject can be human, pig, seal, chicken, quail, goose or duck. In this regard, the compound of the present application can also be used as veterinary drug.

The pharmaceutical composition provided in the present application can comprise a compound described herein, or a prodrug or pharmaceutically acceptable salt thereof, or other pharmaceutically acceptable form thereof, and optionally a pharmaceutically acceptable excipient. In certain embodiments, these compositions optionally further comprise one or more other therapeutic agent(s). Alternatively, the compound of the present application can be administered to a patient in need in combination with one or more other therapeutic agent(s). For example, in treatment against influenza viruses, other therapeutic agent(s) concurrently administered in combination with the compound of the present application or comprised in a pharmaceutical composition in combination with the compound of the present application can be approved anti-inflammatory drug.

The pharmaceutical composition of the present application can be formulated into various dosage forms, such as, but not limited to, emulsions, microemulsions, solutions, suspensions, syrups, elixirs, capsules, tablets, pills, powders and granules; sprays for inhalation administration; and injection preparations for injection.

The term “therapeutically effective amount” refers to an amount sufficient to achieve effective treatment when administered to a patient in need of such treatment. A therapeutically effective amount can vary depending on the subject to be treated and condition of the disease, degree of the disease, and administration route, and can be routinely determined by one of ordinary skill in the art.

In some embodiments, the present application provides a method for screening a compound having the effect of inhibiting influenza B virus infection, which comprises the following steps:

(a) modeling the 3-dimensional structure of influenza B virus nucleoprotein using a 3-dimensional molecular structure modeling software;
(b) screening a candidate compound capable of interacting with phenylalanine at position 209 of influenza B virus nucleoprotein using a molecular docking software; and
(c) verifying whether the candidate compound can inhibit replication of influenza B virus at the cellular level;
wherein, the sequence of influenza B virus nucleoprotein is PDB ID: 3TJ0 in the PDB database.

In step (a), the 3-dimensional molecular structure modeling software can be preferably pyMol software. In step (b), the molecular docking software can be preferably Dock software.

The methods for verifying whether the candidate compound can inhibit the replication of influenza B virus at the cellular level are known in the art. In one embodiment, verifying whether the candidate compound can inhibit the replication of influenza virus can be performed by the following method:

(i) infecting mammalian cells (such as A549 cells or MDCK cells) with influenza B virus at MOI of 0.01 to 1;
(ii) adding the candidate compound to the cell culture after infection for 1 h;
(iii) collecting the supernatant of the cell culture medium every 12 h within 12 h to 72 h after infection; and
(iv) detecting virus content in the collected supernatant using plaque technology;
when the virus content is less than 10³ PFU after 72 hours, the candidate compound is considered to have the effect of inhibiting the replication of influenza B virus.

The present application also provides the compound screened by the above method.

Embodiments of the aspects described herein can be illustrated by the following numbered paragraphs:

1. A method for treating a subject infected with influenza A virus, characterized in administering a therapeutically effective amount of a pharmaceutical composition to the subject, the pharmaceutical composition comprising a compound which inhibits interaction between tyrosine at position 148 of influenza A virus nucleoprotein and CRM1.

2. A method for inhibiting the nuclear export of influenza A virus nucleoprotein in a subject infected with influenza A virus, characterized in administering to the subject a compound which interacts with tyrosine at position 148 of influenza A virus nucleoprotein.

3. The method of paragraph 1 or 2, characterized in that, the subject is a mammal or avian species, preferably human, pig, chicken, quail, goose or duck.

4. The method of any one of paragraphs 1 to 3, characterized in that, the compound is selected from the group consisting of naproxen, naproxen sodium, DL-naproxen, naproxen methyl ester, 23979-41-1, naproxcinod, naproxen-ETEMESIL, naproxen ethyl ester, naproxen glucuronide, 5-chloro naproxen, and 5-iodo naproxen.

5. A method for inhibiting the nuclear export of influenza A virus nucleoprotein, characterized in inhibiting interaction between tyrosine at position 148 of influenza A virus nucleoprotein and CRM1.

6. Use of a compound which inhibits interaction between tyrosine at position 148 of influenza A virus nucleoprotein and CRM1, in the preparation of a medicament for treating a subject infected with influenza A virus.

7. Use of a compound which inhibits interaction between tyrosine at position 148 of influenza A virus nucleoprotein and CRM1, in the preparation of a medicament for inhibiting the nuclear export of influenza A virus nucleoprotein in a subject infected with influenza A virus.

8. The use of paragraph 6 or 7, characterized in that, the subject is a mammal or avian species, preferably human, pig, chicken, quail, goose or duck.

9. The use of any one of paragraphs 6 to 8, characterized in that, the compound is selected from the group consisting of naproxen, naproxen sodium, DL-naproxen, naproxen methyl ester, 23979-41-1, naproxcinod, naproxen-ETEMESIL, naproxen ethyl ester, naproxen glucuronide, 5-chloro naproxen, and 5-iodo naproxen.

10. A medicament for treating a subject infected with influenza B virus, the medicament comprising a compound inhibiting the activity of influenza B virus nucleoprotein by interacting with phenylalanine at position 209 of influenza B virus nucleoprotein.

11. The medicament of paragraph 10, characterized in that, the compound inhibits the interaction between phenylalanine at position 209 of influenza B virus nucleoprotein and CRM1.

12. The medicament of paragraph 10 or 11, characterized in that, the subject is mammal, preferably human or seal.

13. The medicament of any one of paragraphs 10 to 12, characterized in that, the compound is selected from the group consisting of naproxen, naproxen sodium, DL-naproxen, naproxen methyl ester, 23979-41-1, naproxcinod, naproxen-ETEMESIL, naproxen ethyl ester, naproxen glucuronide, 5-chloro naproxen, and 5-iodo naproxen.

14. A method for treating a subject infected with influenza B virus, comprising administering to the subject a therapeutically effective amount of a pharmaceutical composition, the pharmaceutical composition comprising a compound which interacts with phenylalanine at position 209 of influenza B virus nucleoprotein.

15. A method for inhibiting influenza B virus infection in a subject, characterized in administering to the subject a compound which interacts with F209 of influenza B virus nucleoprotein.

16. The method of paragraph 14 or 15, characterized in that, the subject is mammal, preferably human or seal.

17. A method for inhibiting the nuclear export of influenza B virus nucleoprotein, characterized in blocking interaction between phenylalanine at position 209 of influenza B virus nucleoprotein and CRM1.

18. The method of any one of paragraphs 14 to 17, characterized in that, the compound inhibits the interaction between phenylalanine at position 209 of influenza B virus nucleoprotein and CRM1.

19. The method of paragraph 18, characterized in that, the compound is selected from the group consisting of naproxen, naproxen sodium, DL-naproxen, naproxen methyl ester, 23979-41-1, naproxcinod, naproxen-ETEMESIL, naproxen ethyl ester, naproxen glucuronide, 5-chloro naproxen, and 5-iodo naproxen.

20. Use of a compound which interacts with phenylalanine at position 209 of influenza B virus nucleoprotein, in the preparation of a medicament for treating a subject infected with influenza B virus.

21. Use of a compound which interacts with phenylalanine at position 209 of influenza B virus nucleoprotein, in the preparation of a medicament for inhibiting the nuclear export of influenza B virus nucleoprotein in a subject infected with influenza B virus.

22. The use of paragraph 20 or 21, characterized in that, the subject is mammal, preferably human or seal.

23. The use of any one of paragraphs 20 to 22, characterized in that, the compound inhibits the interaction between phenylalanine at position 209 of influenza B virus nucleoprotein and CRM1.

24. The use of paragraph 23, characterized in that, the compound is selected from the group consisting of naproxen, naproxen sodium, DL-naproxen, naproxen methyl ester, 23979-41-1, naproxcinod, naproxen-ETEMESIL, naproxen ethyl ester, naproxen glucuronide, 5-chloro naproxen, and 5-iodo naproxen.

25. Use of phenylalanine at position 209 of influenza B virus nucleoprotein as a target for anti-influenza B virus drug.

26. A method for screening a compound having an effect of inhibiting influenza B virus infection, comprising the following steps:

(a) modeling the 3-dimensional structure of influenza B virus nucleoprotein using a 3-dimensional molecular structure modeling software;
(b) screening a candidate compound capable of interacting with phenylalanine at position 209 of influenza B virus nucleoprotein using a molecular docking software; and
(c) verifying whether the candidate compound can inhibit replication of influenza B virus at a cellular level;
wherein, the sequence of influenza B virus nucleoprotein is PDB ID: 3TJ0 in the PDB database.

27. The method of paragraph 26, wherein the step (c) is performed as follows:

(i) infecting mammalian cells with influenza B virus at MOI of 0.01 to 1;
(ii) adding the candidate compound to the cell culture after infection for 1 hour;
(iii) collecting the supernatant of the cell culture medium every 12 h within 12 h to 72 h after infection; and
(iv) detecting the virus content in the collected supernatant using plaque technology; when the virus content is less than 10³ PFU after 72 hours, the candidate compound is considered to have the effect of inhibiting the replication of influenza B virus.

28. The method of paragraph 26 or 27, wherein in step (a), the 3-dimensional molecular structure modeling software is pyMol software.

29. The method of any one of paragraphs 26 to 28, wherein in step (b), the molecular docking software is Dock software.

30. A compound screened by the method of any one of paragraphs 26 to 29.

### EXAMPLES

The following examples are used for better understanding the present application, but not intended to limit the present application. Unless otherwise specified, the experimental methods in the following examples are conventional methods. Unless otherwise specified, the experimental materials used in the following examples were purchased from conventional biochemical reagent stores. 3 repeated experiments were set up in all quantitative experiments in the following examples, and the results were from the average. Female BALB/c mice were from Beijing Vital River Laboratory Animal Technology Co., Ltd.; and the female BALB/c mice are referred to as mice for short in the following.

### Example 1 Effect of the Residue Y148 of Influenza A Virus Nucleoprotein on the Nuclear Export of ANP

I. Vector Construction

Construction of the wild-type influenza A virus nucleoprotein (ANP) and its mutant with residue Y148 mutation:

The wild-type ANP has the amino acid sequence set forth in NCBI No. ACF54602.1; the mutant fragments of A-Y148A and A-Y148F are the sequences in which the amino acid at position 148 of the wild-type ANP sequence is mutated to alanine and phenylalanine, respectively.

The above wild-type ANP was cloned into pcDNA4TO (Invitrogen, Cat. No. V103020), pCMV-MYC (Clontech, Cat. No. 635689), and the expression vector pHH21 (a kind gift from Professor Yoshihiro Kawaoka, University of Wisconsin-Madison, USA, described in “Neumann G, et al., Generation of influenza A viruses entirely from cloned cDNAs, Proc Natl Acad Sci US A. 1999; 96 (16): 9345-50”). pHH21 and pcDNA4TO belong to the reverse genetic packaging systems for influenza viruses, and the vector pHH21 has the promoter and terminator of RNA polymerase I, for expressing the 8-segment viral RNAs of influenza viruses. pcDNA4TO (Invitrogen, Cat. No. V103020) has the promoter CMV/TO, for expressing the viral protein in the viral ribonucleoprotein complex initiating the virus rescue process, including PA, PB1, PB2, and NP. When the wild-type ANP was cloned into pCMV-MYC, ANP tagged with MYC was expressed.

E. coli DH5a (Invitrogen, Cat. No. 18265017) was used as a cloning host to amplify plasmids. According to instructions for QuickChange Site-Directed Mutagenesis Kit (Stratagene, Cat. No. 200518), tyrosine at position 148 of ANP was respectively mutated to alanine (Y148A) or phenylalanine (Y148F) by site-directed mutagenesis, to give two mutants of ANP: Y148A mutant (hereinafter referred to as A-Y148A) and Y148F mutant (hereinafter referred to as A-Y148F). The side chain of the amino acid residue at position 148 of the mutant A-Y148A does not have a benzene ring structure. The amino acid residue at position 148 of the mutant A-Y148F is phenylalanine, and phenylalanine and tyrosine are aromatic amino acids and phenylalanine has similar structure to tyrosine.

Using a Qiangen kit, the plasmids carrying the wild-type ANP, or the mutant fragments of A-Y148F or A-Y148A were extracted, respectively.

Construction of vectors expressing FLAG-CRM1 and FLAG-importin-α1: CRM1 (having the amino acid sequence of NCBI accession No. CAA69905.2) or importin α1 (having the amino acid sequence of NCBI accession No. AAH67848.1) were cloned into the expression vector pcDNA3-FLAG (addgene, Cat. No. 20011) respectively, and importin-α1 or CRM1 expressed by this vector has FLAG tag.

II. Effects of the Residue Y148 of Influenza A Virus Nucleoprotein on Cell Localization of ANP

The sterilized cover slides were placed in a 24-well plate, one per well. 0.5 mL of 293T cell suspension (1×10⁵ cells/mL) (Shanghai Bogoo Biotechnology Co., Ltd., Item No. BG0021) was added to each well of the 24-well plate, and the culture medium was DMEM medium (ThermoFisher, Cat. No. 11965118) containing 10 wt % fetal bovine serum (FCS) (ThermoFisher, Cat. No. 16000044). The cells were cultured to confluence of 90% to 95% in an incubator (37° C., 5% CO₂). The cell culture medium was replaced with Opti-MEM (ThermoFisher, Cat. No. 51985-042) 2 hours before transfection. The constructed vectors (pHH21 plasmids with the wild-type ANP, the mutant fragment of A-Y148A or the mutant fragment of A-Y148F) and Lipofectamine™ 2000 (Invitrogen, Cat. No. 11668-019) were diluted respectively with Opti-MEM medium at a mass ratio of 1:3 and mixed, and the plasmids were mixed with Lipofectamine™ 2000 after 5 min; after still standing at room temperature for 15 min, the mixtures were added to the cell culture in the wells, and the 24-well plate was gently shaken to mix them with the culture in the wells. The concentration of the vector added was 1 μg/m L. The 24-well plate was returned to the incubator, and the medium was changed to DMEM containing 10 wt % FCS 4-6 h after transfection.

The cover slides were taken at 12, 24, and 36 hours after transfection, subjected to removal of the culture medium by pipetting and washed once with PBS. The cover slides then were fixed with 4% paraformaldehyde for more than 30 min at room temperature or overnight at 4° C. Paraformaldehyde fixing solution was discarded and the cover slides were washed 3 times with PBST (PBS containing 0.5 v/v % Tween) for more than 15 min. Subsequently, the cover slides were blocked with 4 wt % bovine serum albumin (BSA) formulated with PBST at 4° C. overnight or at 37° C. for 1 hour. The blocked cover slides were taken and added with primary antibody: anti-ANP polyclonal antibodies (for the preparation method and sequence of the ANP polyclonal antibodies, please see Liu X, Sun L, Yu M, Wang Z, Xu C, Xue Q, et al., Cyclophilin A interacts with influenza A virus M1 protein and impairs the early stage of the viral replication, Cellular microbiology. 2009; 11 (5): 730-41) formulated with blocking solution, allowing them to bind at 37° C. for 1 hour. Wherein, the blocking solution contained 1 wt % BSA and 5 wt % skim milk, and was formulated with PBST. The primary antibody of anti-ANP polyclonal antibodies was diluted at 1:2000. The cover slides were washed 5 times with PBST, each for 10 min. TRITC-labeled goat anti-rabbit IgG (Abcam, Item No. ab6718, dilution 1:5000) formulated in blocking solution was added as the secondary antibody, followed by binding at 37° C. for 1 hour. The cover slides were washed 5 times with PBST, each for 10 min. DAPI (formulated with pure water at a volume ratio of 1:5000) was used for staining, and the staining was performed at room temperature for 2-5 minutes. The cover slides were washed 3 times with PBST. Mounting solution (50% glycerol, formulated with PBS) was added dropwise to the glass slides, and the cover slides were inverted on the glass slides, followed by sealing the slides. Observation was performed using a laser confocal fluorescence microscope (Olympus FV500). The excitation and emission wavelengths of DAPI channel were 359 nm and 461 nm, and the excitation and emission wavelengths of TRITC channel were 550 nm and 620 nm, with a magnification of 40×. The experimental results are shown in FIG. 1. ANP WT had a complete nuclear-cytoplasmic shuttle process, and localized mainly in nucleus 12 hours after transfection, simultaneously in cytoplasm and nucleus 24 hours after transfection, and mainly in cytoplasm 36 hours after transfection. Because A-Y148F simulates the natural spatial conformation of tyrosine, its location result was similar to that of WT. However, A-Y148A showed a nuclear retention phenomenon 36 hours after transfection and cannot enter cytoplasm, indicating that A-Y148 is essential for the nucleus export of ANP.

III. Effect of the Residue Y148 of Influenza A Virus Nucleoprotein on the Interaction Between ANP and CRM1/IMPα1

293T cells were cotransfected with pcDNA3-FLAG vector comprising CRM1 or importin-α1 (IMPα1) sequence (expressing FLAG-tagged CRM1 and IMPα1) and pCMV-MYC vector with the wild-type or mutant ANP (expressing the wild-type ANP, the mutant fragment A-Y148A or the mutant fragment A-Y148F with a MYC tag at the end). The vectors of pcDNA3-FLAG and pCMV-MYC plasmids added respectively have the concentration of 3 μg/mL and 1μg/mL, and the transfection method was as described above.

The negative control was 293T cells transfected with pCMV-MYC vector having the wild-type or mutant ANP (expressing the wild-type ANP, the mutant fragment A-Y148A or the mutant fragment A-Y148F with MYC-tag at the end) alone, without transfection with pcDNA3-FLAG-CRM1 or IMPα1.

The cell lysis buffer (1 wt % Triton X-100, 150 mM NaCl, 20 mM HEPES, 10 wt % glycerol, 1 mM EDTA (pH 7.4), 5 mM Na₃VO₄) was added 48 hours after transfection, to lyse the cells at 4° C.; and 5 μL of anti-FLAG M2 beads (Sigma, Item No. A2220) were added thereto, to bind at 4 t for 4 h or overnight. The lysate was centrifuged at 5000 rpm for 2 min and the supernatant was discarded. 1 mL of the cell lysis buffer was added to wash 3 times at 4° C., each for 10 min. Finally, 5× loading buffer was added and the samples were boiled for 5 min. SDS-PAGE electrophoresis and western blot analysis were performed using primary antibodies: anti-c-MYC antibody, anti-FLAG antibody (Sigma-Aldrich, Cat. Nos. C3956 and F7425), and anti-β-actin antibody (Santa Cruz company, Cat. No. sc-1616-R); and secondary antibody: HRP-labeled goat anti-mouse monoclonal antibody (Jackson, Cat. No. 115035003).

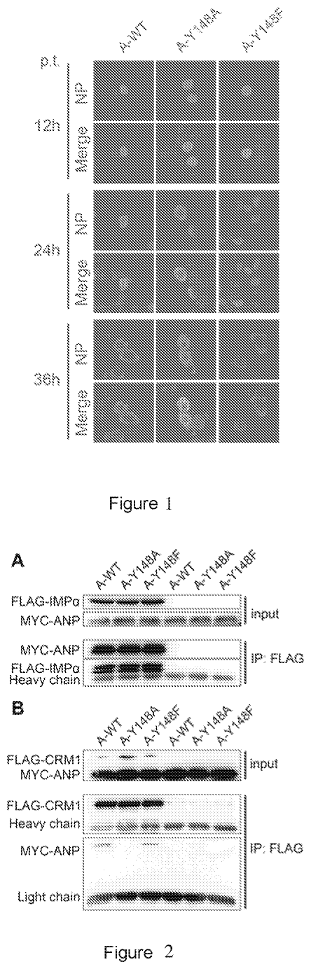

The analysis results are shown in FIG. 2. In each of the images, the samples in lanes 1 to 3 are the samples from the co-transfected cell lysate. The samples in lanes 4 to 6 are the samples from the single-transfected cell lysate (negative control), for removing non-specific interaction between anti-FLAG antibody and other components in the cell lysate. Input represents loading control and IP represents the result of co-immunoprecipitation. Considering that MYC-ANP and FLAG-IMPα have similar molecular weight, they were incubated with each primary antibody (rabbit anti-c-MYC antibody or murine anti-FLAG antibody) in western blot test. In FIG. 2A, FLAG-IMPα represents the western blot result only using the anti-FLAG antibody as the primary antibody; and MYC-ANP represents the western blot result only using the anti-c-MYC antibody as the primary antibody. It can be seen that both Y148 mutations have no effect on the binding of ANP to FLAG-IMPα. In FIG. 2B, FLAG-CRM1 represents the western blot result only using the anti-FLAG antibody as the primary antibody; and MYC-ANP represents the western blot result only using the anti-c-MYC antibody as the primary antibody. The results show that FLAG-CRM1 can merely precipitate the wild-type ANP and the ANP with Y148F mutation, but CRM1-Y148A complex was undetectable, indicating that Y148A mutation causes the loss of the ability of ANP to bind to CRM1.

In summary, both of Y148A and Y148F mutation do not affect the binding of ANP to importin-α1. In other words, Y148 is not the key site for the binding of ANP to IMPα1 (FIG. 2A). However, Y148A mutation inhibits the binding of ANP to CRM1 (FIG. 2B). Y148F mutation in ANP has no effect on the binding of ANP to CRM1, indicating the importance of the benzene ring structure in the side chain of the amino acid at position 148 for functional interaction between ANP and CRM1. These results collectively indicate that the mutant A-Y148A inhibits the nuclear export of ANP by inhibiting the binding of ANP to CRM1.

### Example 2 Residue Y148 in Influenza A Virus Nucleoprotein Affects Replication and Pathogenicity of Influenza A Virus

I. Rescue of Recombinant Influenza a Virus Containing A-Y148 Mutation

Using the method described in Neumann G, Watanabe T, Ito H, Watanabe S, Goto H, Gao P, Hughes M, Perez D R, Donis R, Hoffmann E, Hobom G, Kawaoka Y., Generation of influenza A viruses entirely from cloned cDNAs, Proc Natl Acad Sci USA. 1999, 96 (16): 9345-50, the rescue of the recombinant influenza A virus was performed by replacing the related vector containing NP with the pHH21 vector having the wild-type fragment A-ANP, the mutant fragment A-Y148A or the mutant fragment A-Y148F prepared in Example 1. Transfection was performed with Lipofectamine™ 2000. After transfection for 6 hours, the medium was changed to serum-free DMEM containing 2 μg/ml of TPCK-treated trypsin (SIGMA, Item No. T1426). After culture for 72 to 96 hours, the supernatant was harvested.

II. Detection of Virus Titer by Plaque Assay

The cell culture medium was changed to Opti-MEM (ThermoFisher, Cat. No. 51985-042) in the first 2 hours. The constructed vectors (pHH21 plasmids with the wild-type ANP, the mutant fragment A-Y148A or the mutant fragment A-Y148F) and Lipofectamine™ 2000 (invitrogen, Cat. No. 11668-019) were diluted respectively with Opti-MEM medium at a mass ratio of 1:3 and mixed, and the plasmids were mixed with Lipofectamine™ 2000 after 5 min; after still standing at room temperature for 15 min, the mixtures were added to the cell culture, and the culture vessel was gently shaken to mix them with the culture medium in the wells. The concentration of the vectors added was 0.4 μg/mL. The 24-well plate was returned to the incubator, and the medium was changed to DMEM medium (ThermoFisher, Cat. No. 11965118) containing 10 wt % FCS 4-6 h after transfection.

1 mL of 1×10⁵ cells/mL MDCK cells (Cobioer, Cat. No. CBP60561) was seeded into a 12-well plate, and the culture medium was DMEM medium containing 10 wt % fetal bovine serum FCS (ThermoFisher, Cat. No. 16000044). The cells were incubated in an incubator (37° C., 5% CO₂) overnight, to allow the cells to grow into a monolayer (with confluence of 80% or more). The cells were washed 3 times with PBS, and viruses in different dilutions (A-WT, A-Y148A and A-Y148F) were added to the 12-well plate, 3 parallel wells for each dilution and 1 mL each well, and normal cell control without virus was set up simultaneously. The cells were incubated in the incubator at 37° C. and 5% CO₂ for 1 hour. The virus solution was pipetted, and the cells were washed 3 times with PBS and then the remaining liquid was removed as much as possible. 3 wt % low melting point agarose prepared in ultrapure water was heated to melt, cooled down to about 50° C., mixed with phenol red-free DMEM culture medium preheated at 37° C. at a volume ratio of 1:1 (DMEM containing 4 μg/mL trypsin, i.e., the final concentration of 2 μg/mL), and then quickly added into the 12-well plate, 1 mL per well. The culture plate was placed at 4° C. for about 15 min, and inverted in a 37° C., 5% CO₂ incubator after agarose had been solidified. After culturing for 4 days, the number of plaques was counted and the virus titer was calculated.

The experimental results are shown in Table 1.

TABLE 1
Virus titers measured by plaque assay

| Type of virus | Virus titer 96 h post-transfection: mean ± standard |
|---|---|
| nucleoprotein | deviation (10⁵ PFU/mL) |
| A-WT | 1.78 ± 0.24 |
| A-Y148A | Not detected |
| A-Y148F | 0.44 ± 0.06 |

It can be seen that Y148A mutation of influenza A virus nucleoprotein caused failure of the rescue of viruses, however Y148F mutant virus was successfully rescued, with slightly lower virus titer than A-WT. This indicates that the benzene ring of the aromatic amino acid at position 148 of influenza A virus nucleoprotein is essential for proliferation of influenza A virus.

III. Effect of Y148F Mutation on the Growth Curve of Influenza A Virus

1×10⁸ cells/mL A549 cells (Sigma, Item No. 86012804) were seeded into a 10 cm culture dish, and the culture medium was DMEM medium (ThermoFisher, Cat. No. 11965118) containing 10 wt % fetal bovine serum FCS (ThermoFisher, Cat. No. 16000044). The cells were incubated in an incubator (37° C., 5% CO₂) overnight to grow into a monolayer (with confluence of 80% or more). The cells were washed 3 times with PBS, and the above rescued influenza A virus A-WT or A-Y148F (MOI=0.001) were added to the culture dish. The cells were incubated in a 37° C., 5% CO₂ incubator for 1 hour, the virus solution was pipetted and replaced with DMEM containing 10 wt % FCS, the cell supernatant at different time points was collected for plaque assay (the steps were as described above), the virus titer was detected, and the growth curves for virus were drawn.

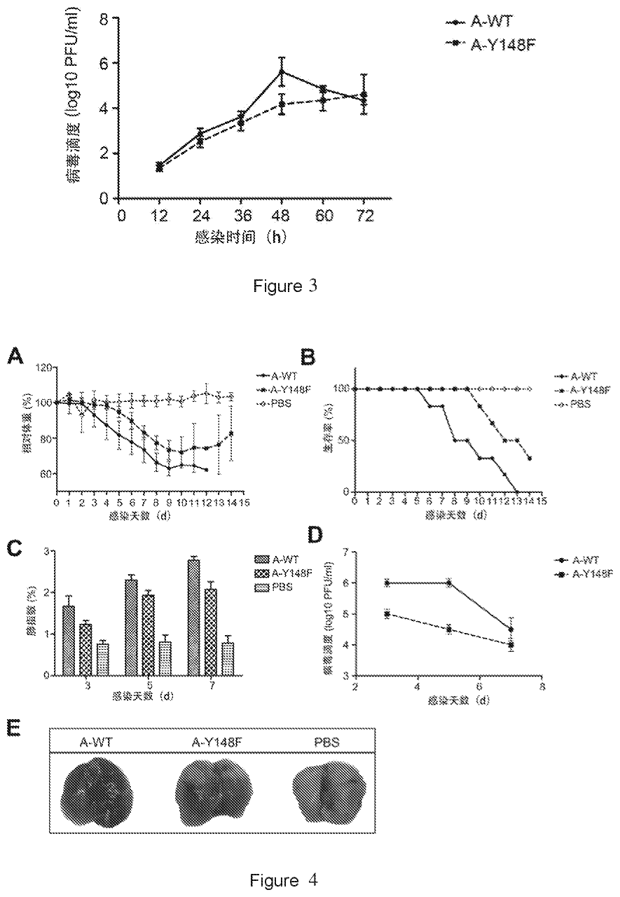

The experimental results are shown in FIG. 3. A-Y148F has no significant difference in the virus titer compared to A-WT 72 hours after virus infection, but has a significant transient decrease in the virus titer at 48 hours post-infection. These results indicate that Y148F mutation has little effect on the proliferation of influenza A virus.

IV. Effect of A-Y148F Mutation on the Replication and Pathogenicity of Virus in Mice

1. Grouping of Mice

Six-week-old female BALB/c mice were anesthetized with diethyl ether, and inhaled with 50 μL (10⁴ PFU/mL) of WSN wild-type or A-Y148F mutant virus by nasal aspiration. The negative control was set to inhale sterilized PBS. Mice were divided into the body weight groups and the dissection groups: the body weight groups were used to observe the body weight and the mortality of mice daily, with 8 mice in each group. The dissection groups were used to dissect, to observe lungs and measure the viral load in the lung tissue, with 9 mice in each group.

2. Detection on Body Weight

The body weight of the mice in the body weight groups was measured and recorded daily, and the pathological features were observed and the number of deaths was recorded for 14 days.

3. Determination on Lung Index

In the dissection groups, 3 mice in each group were dissected on Day 3, Day 5, and Day 7 after infection. The body weight and lung weight of the mice were measured to calculate the lung index (lung index=lung mass/body weight of mouse).

4. Measurement of the Viral Load of the Lung Tissue

For the lungs of the mice from the dissection groups, the lung tissue was homogenized in 1 ml of the ice-cold PBS using QIAGEN TissueLyser II, 30 cycles/s for 4 min, and the homogenate was centrifuged at 5000 g for 10 min. The virus titer was determined by using the plaque assay as described above.

The experimental results are shown in FIG. 4. Compared with PBS group, the body weight of the mice from the WSN WT group (A-WT) was lost rapidly after virus infection, while the body weight of the mice from the A-Y148F group was lost relatively less (FIG. 4A). All mice from the WSN WT group died 13 days after infection, while the survival rate of the mice from the A-Y148F group was 37.5% (FIG. 4B). Influenza viruses mainly infect lung, and we found that 5 days after virus infection, the mice from both WSN WT and A-Y148F groups had obvious macroscopical lesions in the lungs (FIG. 4E), and significantly increased lung index (FIG. 4C) and virus titer in the lungs (FIG. 4D), and the replication ability and pathogenicity of A-Y148F were relatively weaker, but there was no statistically significant difference with WSN WT. The results of the in vivo experiments also indicate that the benzene ring at position 148 of ANP is the key site affecting the proliferation ability of influenza A virus, which is consistent with the in vitro results.

### Example 3 Effect of the Residue F209 of Influenza B Virus Nucleoprotein on the Nuclear Export of Influenza B Virus Nucleoprotein

I. Study on the Key Sites for the Nuclear Export of Nucleoprotein B (BNP)

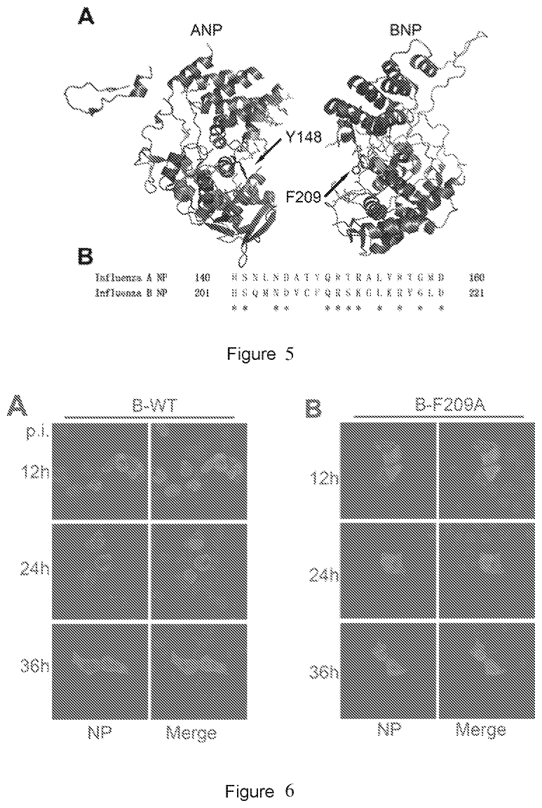

BNP and ANP have very similar structure, and in particular, both contain an RNA-binding groove enriching basic amino acids (Ye Q, Krug R M, Tao Y J., The mechanism by which influenza A virus nucleoprotein forms oligomers and binds RNA, Nature. 2006; 444 (7122): 1078-82; Ng A K, Lam M K, Zhang H, Liu J, Au S W, Chan P K, et al., Structural basis for RNA binding and homo-oligomer formation by influenza B virus nucleoprotein, Journal of virology. 2012; 86 (12): 6758-67). Sequence alignment results show that both ANP and BNP have an aromatic amino acid in the RNA binding groove, Y148 in ANP and F209 in BNP (FIG. 5A), and the sequences adjacent to the two sites have very high homology (FIG. 5B). Therefore, we speculated that B-F209 might function similarly to A-Y148 during the nuclear export of BNP.

II. Construction of the Wild-Type Influenza B Virus Nucleoprotein (BNP) and its Mutant with F209 Residue Mutation

The wild-type BNP has the amino acid sequence set forth in NCBI No. AAA82969.1; the mutant fragment of B-F209A is the sequence in which the amino acid at position 209 of the wild-type BNP sequence is mutated to alanine.

According to the method described in Example 1, the wild-type BNP was cloned into pCMV-MYC vector (Clontech, Cat. No. 631601). E. coli DH5a was used as a cloning host to amplify plasmids. According to the instructions of QuickChange Site-Directed Mutagenesis Kit (Stratagene, Cat. No. 200518), phenylalanine at position 209 of BNP was mutated to alanine (F209A) by site-directed mutagenesis, to give the mutant B-F209A. The corresponding side chain in the mutant has no benzene ring structure. The pCMV-MYC plasmid with the wild-type BNP and the pCMV-MYC plasmid with the mutant B-F209A were extracted separately.

According to the method in Example 1, the expression vectors containing FLAG-CRM1 and FLAG-importin-α1 were constructed.

III. Effect of the Amino Acid Residue F209 of Influenza B Virus Nucleoprotein on Cell Localization of BNP

Using the same method as in Example 1, 293T cells were transfected with the pCMV-MYC plasmid with the wild-type BNP or the mutant fragment of B-F209A, respectively.

Samples were collected at 12, 24, and 36 hours after transfection, and the cover slides were fixed and blocked using the method similar to Example 1. The blocked cover slides were taken, and added with primary antibody: BNP antibody (Santa Cruz, Item No. sc-80483, 1:2000 dilution) formulated with blocking solution (1 wt % BSA, 5% skim milk, prepared with PBST), allowing them to bind at 37° C. for 1 hour. The cover slides were washed 5 times with PBST, each for 10 min. The secondary fluorescent antibody of TRITC-labeled goat anti-rabbit IgG (Abcam, Item No. ab6718, 1:5000 dilution) formulated with the blocking solution was added and bound at 37° C. for 1 hour. The cover slides were washed 5 times with PBST, each for 10 min. Staining was performed with DAPI (formulated at a volume ratio of 1:5000 with pure water) at room temperature for 2-5 minutes. Subsequently, the cover slides were washed 3 times with PBST. Mounting solution (50% glycerol, formulated with PBS) was added dropwise to the glass slides, and the cover glasses were inverted on the glass slides, followed by sealing the surrounding of the slides. Observation was performed using a laser confocal fluorescence microscope (Olympus FV500). The excitation and emission wavelengths of DAPI channel were 359 nm and 461 nm, and the excitation and emission wavelengths of TRITC channel were 550 nm and 620 nm, with a magnification of 40×.

The experimental results are shown in FIG. 6. BNP WT had a complete nuclear-cytoplasmic shuttle process, and localized mainly in the nucleus 12 hours after transfection and simultaneously in cytoplasm and nucleus 24 and 36 hours after transfection (FIG. 6A). However, the mutant of B-F209A localized mainly in nucleus at all 3 time points (FIG. 6B), indicating that the F209A mutation seriously affects the nuclear export process of BNP.

4. Effect of the Residue F209 of Influenza B Virus Nucleoprotein on the Interaction Between BNP and CRM1/IMPα1

Using the same method as in Example 1, 293T cells were co-transfected with pcDNA3-FLAG vector comprising CRM1 or importin-α1 (IMPα1) sequence (expressing FLAG-tagged CRM1 and IMPα1) and the pCMV-MYC vector with the wild-type or mutant ANP (expressing the wild-type ANP, the mutant fragment of A-Y148A or the mutant fragment of A-Y148F with a MYC tag at the end). The negative control was the 293T cells transfected with the pcDNA3-FLAG vector comprising CRM1 or importin-α1 alone.

Using the same method as in Example 1, SDS-PAGE electrophoresis and western blot analysis were performed with primary antibodies: anti-c-MYC antibody, anti-FLAG antibody (Sigma-Aldrich, Cat. Nos. C3956 and F7425), and anti-β-actin antibody (Santa Cruz, Cat. No. sc-1616-R); and secondary antibody: HRP-labeled goat anti-mouse monoclonal antibody (Jackson, Cat. No. 115035003).

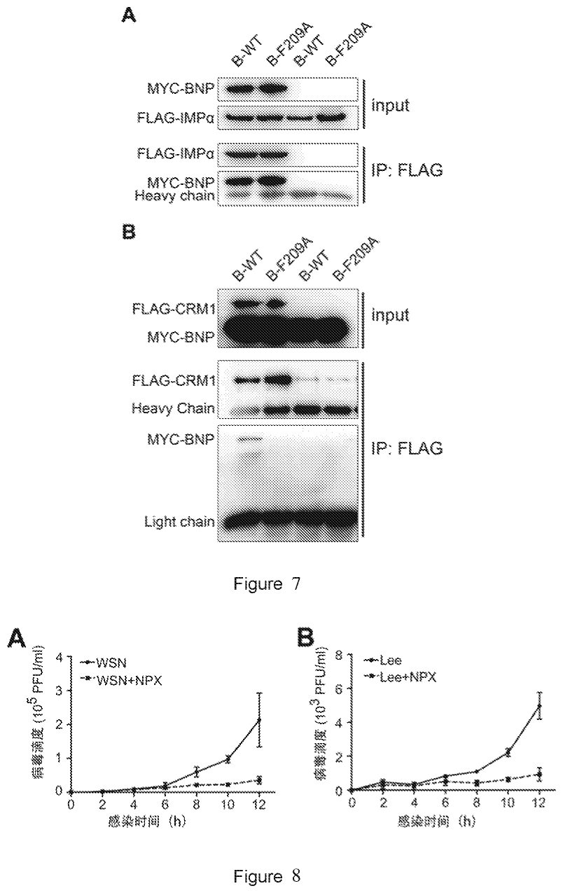

The analysis results are shown in FIG. 7. In each of the images, the samples in lanes 1 to 2 are the samples from the co-transfected cell lysate. The samples in lanes 3 to 4 are the samples from the single-transfected cell lysate (negative control), for removing non-specific interactions between anti-MYC antibody and other components in the cell lysate. Input represents loading control and IP represents the results of co-immunoprecipitation. In FIG. 7A, FLAG-IMPα represents the western blot results only using anti-FLAG antibody as the primary antibody; and MYC-BNP represents the western blot results only using anti-c-MYC antibody as the primary antibody. IP results showed a significant binding of MYC-BNP to FLAG-IMPα (lanes 1-2); while MYC-BNP was not detected in lanes 3-4 for single transfection with MYC-BNP. It indicates that B-F209A mutation does not affect the binding of BNP to IMPα. In FIG. 7B, FLAG-CRM1 represents the western blot results only using anti-FLAG antibody as the primary antibody; and MYC-ANP represents the western blot results only using anti-c-MYC antibody as the primary antibody. IP results showed that the wild-type BNP in lane 1 can effectively bind to CRM1, while a weak MYC-BNP band was detected in lane 2, indicating that the F209A mutation reduces the binding of BNP to CRM1.

In summary, the F209A mutation does not affect the binding of BNP to importin-α1. In other words, F209A is not the key site for the binding of BNP to IMPα1 (FIG. 7A). However, the F209A mutation inhibits the binding of BNP to CRM1 (FIG. 7B), indicating the importance of the amino acid at position 209 for functional interaction between BNP and CRM1. These results collectively show that the mutant B-F209A inhibits the nuclear export of BNP by inhibiting the binding of BNP to CRM1.

In combination with the results of ANP and BNP, we believe that the aromatic amino acids A-Y148 and B-F209 in the RNA binding grooves of ANP and BNP are the key sites for regulating the nuclear export of NP.

### Example 4 A-Y148 and B-F209 are the Key Sites for the Binding of Naproxen to ANP/BNP

The interaction between naproxen and ANP/BNP was measured using isothermal titration microcalorimeter (NANO-ITC 2G, TA Instruments). Isothermal titration calorimetry (ITC) can measure the change in heat during the interaction between two molecules. 1 mM naproxen solution and 0.1 mM ANP/BNP solution were prepared with 50 mM potassium phosphate buffer (pH 6.5) containing 1% DMSO, with stirring at room temperature for 15 min. The buffer solution alone was added into the reference cell, 1.48 mL of the ANP or BNP solution was added into the sample cell, and 8 μL of the naproxen solution was contained in the stirring syringe. Each injection of the naproxen solution was performed for 16 s, with a 240 s pause. Each injection can form a thermal pulse, which was integrated with time and then subjected to concentration normalization, to generate a titration curve of kcal/mol vs. molar ratio (ligand/sample). The resulting isotherm was fitted according to binding models, to obtain the affinity (Kd), stoichiometry (n), and enthalpy of interaction (ΔH).

In order to further verify whether the sites A-Y148 and B-F209 are the key sites for the interaction between naproxen with ANP and BNP, the solution of the protein A-Y148F or B-F209A carrying the mutation was titrated separately with naproxen solution according to the above method, and the generated thermodynamic parameters were compared with the ANP/BNP solution. The experimental results are shown in Table 2.

TABLE 2
Thermodynamic parameters measured by ITC

| Protein | Kd(M) | n | ΔH(kJ/mol) | ΔS(J/mol · K) |
|---|---|---|---|---|
| ANP | 4.37 × 10⁻⁵ | 1.23 | −5.57 | 64.76 |
| ANP Y148F | 2.25 × 10⁻⁴ | 0.10 | −45.70 | −83.45 |
| BNP | 3.42 × 10⁻⁸ | 0.93 | −1.36 | 138.40 |
| BNP F209A | 3.30 × 10⁻⁶ | 1.68 | −0.70 | 102.6 |

The Kd value of naproxen-ANP/BNP shows the interaction between naproxen and ANP/BNP. It should be noted that the interaction between naproxen and BNP is significantly stronger than that of naproxen and ANP. Moreover, all the mutations at Y148 of BNP and F209 of BNP result in decreased binding of naproxen to ANP and BNP, indicating that A-Y148 and B-F209 are the key sites for the binding of naproxen to ANP/BNP.

### Example 5 Effects of Naproxen on Replication of Influenza A and B Viruses in Cells

I. Effect of Naproxen on the Growth Curves of Influenza A and B Viruses

1×10⁸ cells/mL of A549 cells (Sigma, Item No. 86012804) were seeded into 10 cm culture dishes, and the culture medium was DMEM medium (ThermoFisher, Cat. No. 11965118) containing 10 wt % fetal bovine serum FCS (ThermoFisher, Cat. No. 16000044). The cells were incubated in an incubator (37° C., 5% CO₂) overnight to grow to a monolayer (with confluence of 80% or more). The cells were washed 3 times with PBS, and the above rescued influenza A virus WSN A/WSN/1933 (H1N1) (Maorong Yu, Xiaoling Liu, Shuai Cao, et al., Identification and Characterization of Three Novel Nuclear Export Signals in the Influenza A Virus Nucleoprotein. Journal of Virology, 2012 (86): 4970-4980) (MOI=0.1) or influenza B virus Lee40 (Cao S, Jiang J, Li J, Li Y, Yang L, Wang S, Yan J, Gao G F, Liu W. Characterization of the nucleocytoplasmic shuttle of the matrix protein of influenza B virus. J Virol. 2014; 88 (13): 7464-73. Doi: 10.1128/JVI.00794-14) (MOI=1) was added to the culture dishes. The cells were incubated in an incubator at 5% CO₂ and 37° C. for 1 hour. The virus solution was removed by pipetting, the cells were washed 3 times with PBS and then the remaining liquid was removed as much as possible, and a normal cell medium (DMEM containing 10 wt % FCS) containing 12 ng of naproxen (Sigma, Item No. 82170) was added to each dish. The cell supernatant was collected at different time points for subsequent plaque assay (in which the steps are as described in Example 2) to detect the virus titers, and the virus growth curves were drawn.

The experimental results are shown in FIG. 8. Compared to the cells from the untreated group, the naproxen-treated (+NPX) cells showed a significant decrease in virus titer, indicating the antiviral effects of naproxen against both influenza A (FIG. 8A) and B viruses (FIG. 8B).

II. Effects of Naproxen on the Nuclear Export of ANP and BNP

The sterilized cover slides were placed in a 24-well plate, one per well. 1×10⁵ cells/mL of A549 cell suspension was added to each well, and the culture medium was DMEM containing 10 wt % FCS. The cells were cultured in an incubator (37° C., 5% CO₂) to confluence of 90% to 95% and then washed 3 times with PBS, and the rescued influenza A virus WSN A/WSN/1933 (H1N1) (MOI=0.1) and influenza B virus Lee40 (MOI=1) were added to the 24-well plate. The cells were incubated in an incubator at 37° C. and 5% CO₂ for 1 hour, the virus solution was removed by pipetting, the cells were washed 3 times with PBS, and then the remaining liquid was removed as much as possible. A normal cell culture medium (DMEM containing 10 wt % FCS) containing 400 μg naproxen was added to each well of the experimental group. Naproxen was not added to the wells of the control group.

The culture medium was removed by pipetting 12 hours after virus infection, and the cover slides were fixed, blocked, and detected by immunofluorescence according to the steps described in Example 1. The primary antibody used was ANP polyclonal antibodies (please see Liu X, Sun L, Yu M, Wang Z, Xu C, Xue Q, et al., Cyclophilin A interacts with influenza A virus M1 protein and impairs the early stage of the viral replication, Cellular microbiology. 2009; 11 (5): 730-41) or BNP antibody (Santa Cruz, Cat. No. sc-80483), and the fluorescent secondary antibody used was TRITC-labeled goat anti-rabbit IgG. Fluorescence location in the DAPI and TRITC channels was observed using a laser confocal fluorescence microscope (Olympus FV500). The excitation and emission wavelengths of DAPI were 359 nm and 461 nm, and the excitation and emission wavelengths of TRITC were 550 nm and 620 nm, with a magnification of 40×.

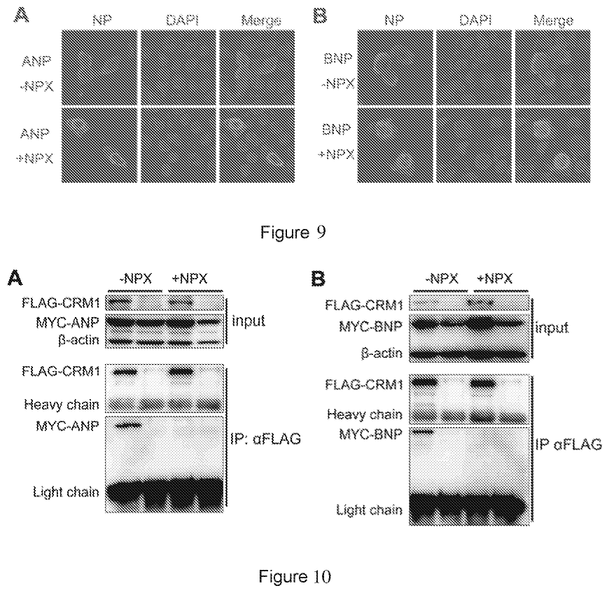

The experimental results are shown in FIG. 9. 24 hours after virus infection, ANP (FIG. 9A) and BNP (FIG. 9B) localized mainly in the nucleus in the naproxen-treated cells, while mainly in the cytoplasm in untreated group, indicating that naproxen can significantly inhibit the nuclear export of ANP and BNP.

III. Effects of Naproxen on the Binding of ANP and BNP to CRM1

3×10⁷ cells/mL of 293T cells were seeded into 60 mm culture dishes, and cultured overnight in an incubator (37° C., 5% CO₂), allowing them to grow to a monolayer (with confluence of 90% to 95% or more). The 293T cells were co-transfected with the pCMV-MYC vector comprising the wild-type ANP constructed in Example 1 (expressing ANP with MYC tag) or the pCMV-MYC vector comprising the wild-type BNP constructed in Example 3 (expressing BNP with MYC tag), and the FLAG-CRM1 expression vector constructed in Example 1, and incubated in an incubator at 37° C. and 5% CO₂ for 1 h. The virus solution was removed by pipetting, the cells were washed 3 times with PBS, and then the remaining liquid was removed as much as possible. 4 ng of naproxen was added in each culture dish. The 293T cells transfected with the pCMV-MYC vector comprising the wild-type ANP constructed in Example 1 (expressing ANP with MYC tag) or the pCMV-MYC vector comprising the wild-type BNP constructed in Example 3 (expressing BNP with MYC tag) were used as control.

The cell lysis buffer (1 wt % Triton X-100, 150 mM NaCl, 20 mM HEPES, 10 wt % glycerol, 1 mM EDTA (pH 7.4), 5 mM Na₃VO₄) was added to lyse the cells at 4° C. 48 hours after transfection, and 5 μL of anti-FLAG M2 beads (Sigma, Item No. A2220) was added thereto and bound at 4° C. for 4 h or overnight. The lysate was centrifuged at 5000 rpm for 2 min and the supernatant was discarded. 1 mL of the cell lysis buffer was added to wash 3 times at 4° C., each for 10 min. Finally, 5× loading buffer was added and the samples were boiled for 5 min. SDS-PAGE electrophoresis and western blot analysis were performed using primary antibodies: anti-c-MYC antibody, anti-FLAG antibody (Sigma-Aldrich, Cat. Nos. C3956 and F7425), and anti-β-actin antibody (Santa Cruz company, Cat. No. sc-1616-R); and secondary antibody: HRP-labeled goat anti-mouse monoclonal antibody (Jackson, Cat. No. 115035003).

The analysis results are shown in FIG. 10. In each of the images, the samples in lanes 1 and 3 are the co-transfected cell lysate, and the samples in lanes 2 and 4 are the lysate of the single-transfected negative control, removing the non-specific interactions of anti-FLAG antibodies and other components in the cell lysate. The samples in lanes 3 and 4 are those in which naproxen was added to the cell culture. Input represents the loading control and IP represents the results of co-immunoprecipitation using anti-FLAG M2 beads (aFLAG). FLAG-CRM1 represents the western blot results only using anti-FLAG antibody as the primary antibody; and MYC-ANP (FIG. 10A) or MYC-BNP (FIG. 10B) represents the western blot results only using anti-c-MYC antibody as the primary antibody. In FIGS. 10A and 10B, lane 1 shows that both ANP and BNP can bind to CRM1 in the absent of naproxen. Lane 3 shows that when treated with naproxen, ANP or BNP did not bind to CRM1. Lanes 2 and 4 were used for removing the non-specific interactions of anti-FLAG antibodies and other components.

It can be seen that naproxen inhibited the binding of ANP (FIG. 10A) and BNP (FIG. 10B) to CRM1, indicating that naproxen can inhibit the nuclear export of NP by inhibiting the binding of ANP/BNP to CRM1.

### Example 6 Effects of Naproxen on the Replication and Pathogenicity of Influenza A and B Viruses in Mice

1. Grouping of Mice

Six-week-old BALB/c mice were anesthetized with diethyl ether, and inhaled with 50 μL of influenza A virus WSN A/WSN/1933 (H1N1) (10⁴ PFU/mL) or influenza B virus Lee40 (10⁵ PFU/mL) by nasal aspiration. The negative control was set to inhale sterilized PBS. On Day 1 after the virus infection, the mice were administered with oseltamivir (Sigma, product number PHR1871) (1 mg) via gastric perfusion, 1 mL of low-dose (5 mM) or high-dose (50 mM) naproxen via intraperitoneal injection, or PBS (1 mL) via intraperitoneal injection as control. The mice were divided into the body weight groups and the dissection groups: the body weight groups were used to observe the body weight and mortality of the mice daily, with 8 mice in each group. The dissection groups were used to dissect, for observing lungs and measuring the viral load in the lung tissue, with 12 mice in each group.

2. Body Weight Measurement

The body weight of the mice in the body weight groups was measured and recorded daily, and the pathological features were observed and the number of deaths was recorded for 14 days.

3. Determination of Lung Index

In the dissection groups, 3 mice in each group were dissected on Day 2, Day 4, Day 6 and Day 8 after infection, respectively. The body weight and lung weight of the mice were measured to calculate the lung index (lung index=lung mass/body weight of mice).

4. Measurement of the Viral Load of Lung Tissue

For the lungs of the mice from the dissection groups, the lung tissue was homogenized in 1 mL of the ice-cold PBS using QIAGEN TissueLyser II, 30 cycles/s for 4 min, and the homogenate was centrifuged at 5000 g for 10 min. The virus titer was determined by the plaque assay described in Example 2.

In the dissection groups, 3 mice in each group were dissected on Day 2, Day 4, Day 6 and Day 8 after infection. The body weight and lung weight of the mice were measured to calculate the lung index (lung index=lung mass/body weight of mice).

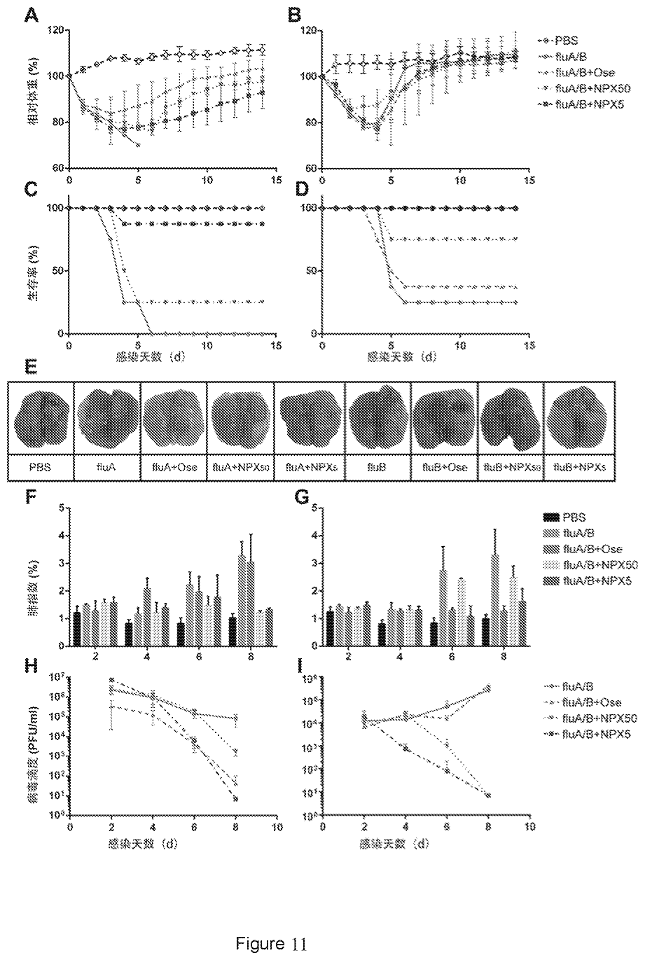

The experimental results are shown in FIG. 11. The mice infected with influenza A virus and injected with PBS (fluA) had rapid body weight loss after infection, and all died on Day 6; and the mice infected with influenza A virus and treated with oseltamivir (fluA+Ose) or naproxen (fluA+NPX50 and fluA+NPX5) had repid body weight loss Days 1 to 4 after viral infection, and then slowly recovered (FIG. 11A, C). The mice infected with influenza B virus and injected with PBS (fluB), and the mice infected with influenza B virus and treated with oseltamivir (fluB+Ose) or naproxen (fluB+NPX50 and fluB+NPX5) had rapid body weight loss Days 1 to 4 after viral infection, and then slowly recovered, and had no significant difference with the PBS group after 1 week (FIG. 11B). The survival rates of the fluA+oseltamivir group (fluB+Ose), the fluA+naproxen high-dose group (fluB+NPX50), and the fluA+naproxen low-dose group (fluB+NPX5) were 100%, 25%, and 87.5%, respectively. Among them, the difference between the high-dose and low-dose groups is significant, with the low-dose group showing a higher survival rate (FIG. 11C). The survival rates of the fluB group, the fluB+oseltamivir group, the fluB+naproxen high-dose group, and the fluB+naproxen low-dose group were 25%, 37.5%, and 75%, 100%, respectively (FIG. 11D). It can be seen that compared to oseltamivir, naproxen has a better therapeutic effect against influenza B virus, in which all mice from the low-dose group survived (FIG. 11D). The data of macroscopical lesions in the lung (FIG. 11E) and the lung index (FIGS. 11F and 11G) shows that in the fluA+oseltamivir group, the fluA+naproxen low dose group, the fluB+naproxen low dose group, and the fluB+naproxen high dose group, lung injuries were milder and lung indexes were lower. The viral load results of the lung show that for fluA, the treatment effects of oseltamivir and low-dose naproxen were better (FIG. 11H), while for fluB, the treatment effects of low-dose and high-dose naproxen were better (FIG. 1).

The above experimental results show that naproxen can inhibit the replication and pathogenicity of influenza A and B viruses in vivo.

1.-30. (canceled)

31. A method for treating a subject infected with influenza virus, comprising administering to the subject a therapeutically effective amount of a pharmaceutical composition, the pharmaceutical composition comprising a compound which inhibits activity of influenza virus nucleoprotein by interacting with an aromatic amino acid in the RNA binding groove of influenza virus nucleoprotein.

32. The method of claim 31, wherein the aromatic amino acid is selected from tyrosine at position 148 of influenza A virus nucleoprotein or phenylalanine at position 209 of influenza B virus nucleoprotein.

33. The method of claim 31, wherein the compound inhibits or blocks interaction between influenza virus nucleoprotein and CRM1.

34. The method of claim 31, wherein the compound is selected from the group consisting of naproxen, naproxen sodium, DL-naproxen, naproxen methyl ester, 23979-41-1, naproxcinod, naproxen-ETEMESIL, naproxen ethyl ester, naproxen glucuronide, 5-chloro naproxen, and 5-iodo naproxen.

35. The method of claim 31, wherein the subject is mammal or avian, preferably human, seal, pig, chicken, quail, goose or duck

36. The method of claim 31, wherein the subject has influenza A virus infection and/or influenza B virus infection.

37. A method for inhibiting the nuclear export of influenza virus nucleoprotein, comprising administering a compound to a subject in need thereof, wherein the compound interacts with an aromatic amino acid in the RNA binding groove of influenza virus nucleoprotein.

38. The method of claim 37, wherein the aromatic amino acid is selected from tyrosine at position 148 of influenza A virus nucleoprotein or phenylalanine at position 209 of influenza B virus nucleoprotein.

39. The method of claim 37, wherein the compound inhibits or blocks interaction between influenza virus nucleoprotein and CRM1.

40. The method of claim 37, wherein the compound is selected from the group consisting of naproxen, naproxen sodium, DL-naproxen, naproxen methyl ester, 23979-41-1, naproxcinod, naproxen-ETEMESIL, naproxen ethyl ester, naproxen glucuronide, 5-chloro naproxen, and 5-iodo naproxen.

41. The method of claim 37, wherein the subject is mammal or avian, preferably human, seal, pig, chicken, quail, goose or duck.

42. The method of claim 37, wherein the subject has influenza A virus infection and/or influenza B virus infection.

43. A method for screening a compound having an effect of inhibiting influenza virus infection, the method comprising:
(a) modeling the 3-dimensional structure of influenza virus nucleoprotein using a 3-dimensional molecular structure modeling software;
(b) screening a candidate compound capable of interact with an aromatic amino acid in the RNA binding groove of influenza virus nucleoprotein using a molecular docking software; and
(c) verifying whether the candidate compound can inhibit replication of influenza virus at a cellular level.

44. The method of claim 43, wherein the aromatic amino acid is selected from tyrosine at position 148 of influenza A virus nucleoprotein or phenylalanine at position 209 of influenza B virus nucleoprotein.

45. The method of claim 43, wherein the sequence of influenza B virus nucleoprotein is PDB ID: 3TJ0 in the PDB database

46. The method of claim 43, wherein the step (c) is performed as follows:
(i) infecting mammalian cells with influenza virus at MOI of 0.01 to 1;
(ii) adding the candidate compound to the cell culture after infection for 1 hour;
(iii) collecting the supernatant of the cell culture medium every 12 h within 12 h to 72 h after infection; and
(iv) detecting the virus content in the collected supernatant using plaque technology;
when the virus content is less than 10³ PFU after 72 hours, the candidate compound is considered to have the effect of inhibiting the replication of influenza virus.

47. The method of claim 43, wherein in step (a), the 3-dimensional molecular structure modeling software is pyMol software.

48. The method of claim 43, wherein in step (b), the molecular docking software is Dock software.

49. A compound screened by the method of claim 43.
